(19) United States
(12) Patent Application Publication
(10) Pub. No.: US 2026/0241192 A1
(43) Pub. Date: Aug. 20, 2026

(54) ELECTROMAGNETIC FREQUENCY GENERATION SYSTEM AND METHOD

(71) Applicant: THE TRUSTEES OF INDIANA UNIVERSITY, Bloomington, IN (US)

(72) Inventors: Felipe P. PEREZ, Indianapolis, IN (US); Maher E. RIZKALLA, Carmel, IN (US)

(21) Appl. No.: 18/869,913

(22) PCT Filed: Jul. 6, 2023
(86) PCT No.: PCT/US2023/027007

§ 371 Date: Nov. 27, 2024

Related U.S. Application Data
(60) Provisional application No. 63/358,627, filed on Jul. 6, 2022.

(51) Int. Cl. A61N 2/00; A61N 2/02

(52) U.S. Cl. CPC A61N 2/006; A61N 2/02

(57) ABSTRACT

The present disclosure relates to an electromagnetic field (EMF) generation system for treatment of neurodegenerative disease in a patient. The EMF generation system includes a head-mounted antenna unit positionable on a patient's head, a signal generator to generate one or more time-varying electrical signals, at least one coupling circuit coupled between the signal generator and the head-mounted antenna unit, and at least one controller coupled to the signal generator.

### PRIORITY CLAIM

This application claims priority under 35 U.S.C. § 119(e) to U.S. Provisional Application No. 63/358,627, filed Jul. 6, 2022, which is expressly incorporated by reference herein in its entirety.

### GOVERNMENT SUPPORT CLAUSE

This invention was made with government support under TR000006 awarded by National Institutes of Health. The government has certain rights in the invention.

### FIELD

The present disclosure relates generally to electromagnetic field (EMF) generation systems and methods. More specifically, the present disclosure relates to electromagnetic field (EMF) generation systems and methods for the treatment of neurodegenerative diseases including but not limited to Alzheimer's disease (AD) Parkinson's disease, Lewy body dementia, Frontotemporal dementia, Primary progressive aphasia.

### BACKGROUND

Alzheimer's disease (AD) is a progressive brain disorder that requires complex treatment. It is the sixth leading cause of death in the United States and the fifth leading cause among those over age 65. AD causes the loss of memory and mental deterioration. It is also the most common cause of dementia associated with a progressive neurodegenerative disorder, with a prevalence of 44 million people in the world in 2015, and this figure is estimated to double by 2050. The cause of AD is the accumulation of a protein called amyloid-β peptide or β amyloid, a consequent decrease in acetylcholine levels, and a reduction of cerebral blood flow.

There have been numerous efforts with pharmacological treatments such as acetylcholinesterase inhibitors and N-methyl-D-aspartate (NMDA) receptor antagonists, and etiology-based treatments such as secretase inhibitors, amyloid binders, and tau therapies. Despite extensive research in the treatment and management of AD, it is highly unlikely that any one drug will successfully treat this disease because of the blood brain barrier and the blood-cerebrospinal fluid (CSF) barrier creates an obstacle for an effective systemic drug delivery to the central nervous system.

Over the past several decades, researchers have investigated the use of radiation as an alternative treatment. While radiation presents the most important diagnostic tools and therapeutic modalities in modern medicine, there are still major issues regarding its beneficial effects on the central nervous system (CNS). Recent research has suggested that low-dose non-ionizing electromagnetic field stimulation could be a valuable therapeutic tool for the treatment of neurodegenerative diseases. Recently, it was found that repeated electromagnetic field stimulation (REMFS) decreases the toxic Q amyloid levels that are elevated in AD due to decreased degradation of this protein. The advantages of electromagnetic fields over pharmacological treatments include (1) it easily crosses the blood-brain barrier, (2) it has intraneuronal effects, (3) it has homogeneous field distribution on all the neurons of the brain, (4) it is not dependent on blood circulation to reach neurons, (5) and it has high bioavailability compared to pharmacologic agents. Accordingly, there is a need for utilizing a non-ionizing, non-invasive, low frequency electromagnetic field (EMF) for the treatment of AD patients via radiofrequency (RF) devices.

### SUMMARY

The present disclosure may comprise one or more of the following features and combinations thereof.

According to a first aspect of the present disclosure, an electromagnetic field (EMF) generation system for treatment of neurodegenerative disease in a patient includes a head-mounted antenna unit, a signal generator for generating one or more time-varying electrical signals, at least one coupling circuit, and at least one controller.

The head-mounted antenna unit has at least one ring arranged about a central axis of the head-mounted antenna unit and a plurality of antenna legs coupled to the at least one ring. A first antenna leg of the plurality of antenna legs is spaced apart 180 degrees from a second antenna leg of the plurality of antenna legs relative to the central axis such that the first antenna leg and the second antenna leg are arranged around a patient's head when the head-mounted antenna unit is positioned on the patient's head.

The at least one coupling circuit is coupled between the signal generator and at least one of the plurality of antenna legs such that each antenna leg is responsive to the one or more time-varying electrical signals generated by the signal generator to produce at least one corresponding time-varying electromagnetic field configured to be directed into the patient's head.

The at least one controller is coupled to the signal generator. The at least one controller includes at least one processor and at least one memory storing instructions to, when executed by the at least one processor, determine a power of the time-varying electrical signals based on measured parameters related to physical characteristics of the patient's head which will result in the at least one time-varying electromagnetic field penetrating the patient's head to a target depth.

In some embodiments, the at least one memory may further include instructions to, when executed by the at least one processor, receive user inputs to obtain the measured parameters related to the physical characteristics of the patient's head.

In some embodiments, the plurality of antenna legs may include sixteen antenna legs.

In some embodiments, the at least one time-varying electromagnetic field may have a frequency of about 10 to about 400 MHz. In some embodiments, the frequency may be about 64 MHz.

In some embodiments, the power of the time-varying electrical signals may cause a specific absorption rate of about 0.4 to about 0.6 W/kg.

In some embodiments, the measured parameters may include at least one of the conductivity, permittivity, and density of one or more features of the patient's head. In some embodiments, dielectric properties of each of the features may be determined using the at least one of the conductivity, permittivity and density values of each of the features for individual EMF frequencies. In some embodiments, the memory may further includes instructions to, when executed by the processor, determine the power of the time-varying electrical signals with at least the dielectric properties.

According to another aspect of the present disclosure, a head-mounted antenna unit for treatment of neurodegenerative disease in a patient includes at least one ring arranged about a central axis, a plurality of antenna legs coupled to and extending axially away from the at least one ring, and at least one wire coupled to at least one antenna leg of the plurality of antenna legs.

A first antenna leg of the plurality of antenna legs is spaced apart 180 degrees from a second antenna leg of the plurality of antenna legs relative to the central axis such that the first antenna leg and the second antenna leg are arranged around a patient's head when the head-mounted antenna unit is positioned on the patient's head.

The at least one wire is configured to electrically communicate with a signal generator to produce at least one corresponding time-varying electromagnetic field configured to be directed into the patient's head from at least one antenna leg of the plurality of antenna legs.

In some embodiments, the plurality of antenna legs may include sixteen antenna legs.

In some embodiments, the at least one time-varying electromagnetic field may have a frequency of about 10 to about 400 MHz. In some embodiments, the frequency may be about 64 MHz.

In some embodiments, the at least one wire may include a first wire and a second wire. In some embodiments, the first wire may be coupled with the first antenna leg and the second wire may be coupled with the second antenna leg. In some embodiments, the first wire and the second wire may be configured to selectively operate the first antenna leg and the second antenna leg.

According to another aspect of the present disclosure, a method of treating neurodegenerative disease for a particular patient includes inputting measured physical parameters of a patient's head into a memory of a treatment system, positioning an antenna unit at least partially about the patient's head including a plurality of antenna legs, determining from the stored parameters of the patient's head a power of electrical signals to be applied to the at least one antenna leg of the antenna unit to cause the at least one antenna leg to produce at least one time-varying electromagnetic field to penetrate a target depth into the patient's skull, and applying the electrical signals with the power for a determined treatment time period.

In some embodiments, the method may further comprise obtaining measurements of the patient's head and determining the measured physical parameters from the measurements of the patient's head.

In some embodiments, the measured parameters may include at least one of the conductivity, permittivity, and density of one or more features of the patient's head. In some embodiments, dielectric properties of each of the layers may be determined using the at least one of the conductivity, permittivity and density values of each of the layers for individual EMF frequencies. In some embodiments, the method may further comprise determining the power of the time-varying electrical signals with at least the dielectric properties.

In some embodiments, the target depth into the patient's skull may be the center of the patient's skull.

In some embodiments, the antenna unit may be a birdcage coil antenna.

In some embodiments, the at least one time-varying electromagnetic field may have a frequency of about 64 MHz.

In some embodiments, the power of the time-varying electrical signals may cause a specific absorption rate of about 0.4 to about 0.6 W/kg.

These and other features of the present disclosure will become more apparent from the following description of the illustrative embodiments.

### BRIEF DESCRIPTION OF THE DRAWINGS

The following description accompanies the drawings, all given by way of non-limiting examples that may be useful to understand how the described method and kit may be embodied.

FIG. 1 is a perspective view of an EMF generation system for treatment of neurodegenerative disease in a patient having a head-mounted antenna unit to be positioned on the patient's head, a control system for generating one or more time-varying electrical signals, and a coupling circuit coupled between the signal generator and the head-mounted antenna to produce at least one corresponding time-varying electromagnetic field directed into the patient's head, and

FIG. 1B is a diagrammatic representation of the control system of FIG. 1A showing that the control system includes a signal generator for generating the one or more time-varying electrical signals and a controller coupled to the signal generator to determine a power of the one or more time-varying electrical signals based on measured parameters related to physical characteristics of the patient's head so that the at least one time-varying electromagnetic filed penetrates the patient's head to a target depth;

FIG. 2 is a perspective view of the head-mounted antenna unit of FIG. TA on the patient's head showing that the head-mounted antenna unit includes at least one ring arranged about a central axis of the head-mounted antenna unit and a plurality of antenna legs coupled to the at least one ring;

FIG. 3 is diagrammatic representation of the EMF generation system of FIG. 1A;

FIG. 4 is a perspective view of the head-mounted antenna unit of FIG. 1A;

FIG. 5 is a schematic of the coupling circuit of FIG. 1A; and

FIG. 6 is an equivalent circuit of the schematic of FIG. 5 illustrating equivalent impedance values Z1, Z2.

### DETAILED DESCRIPTION

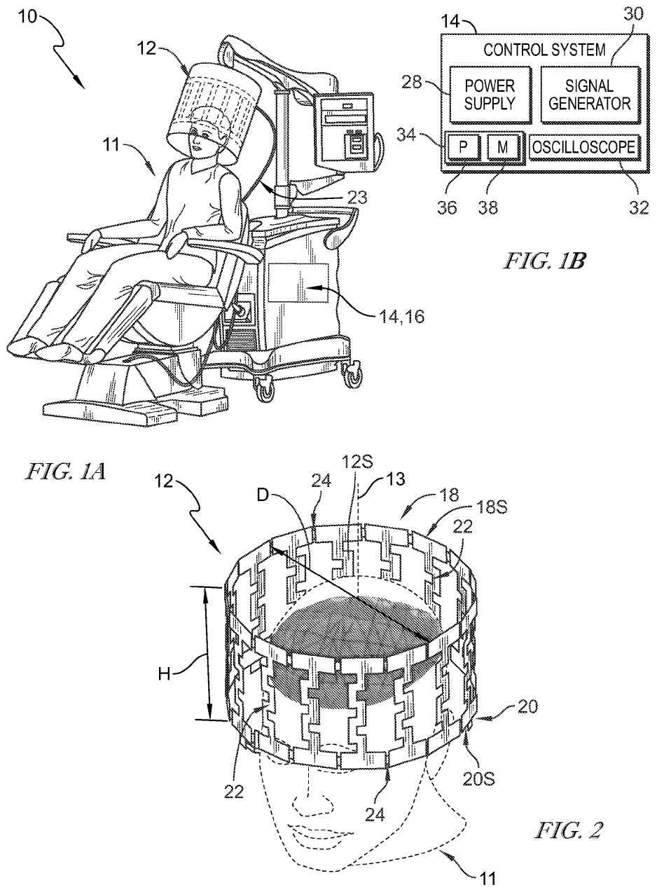

The present disclosure relates to an electromagnetic field (EMF) generation system 10 for treatment of neurodegenerative diseases in a patient 11 as shown in FIG. 1A. Such neurodegenerative diseases include, but are not limited to, Alzheimer's disease (AD) Parkinson's disease, Lewy body dementia, Frontotemporal dementia, and Primary progressive aphasia. The present disclosure further relates to a method of treating neurodegenerative disease for a particular patient. The EMF generation system 10 and corresponding method of treating neurodegenerative disease utilize patient-specific data, such as physical characteristics obtained from a magnetic resonance imaging (MRI) scan, to generate at least one time-varying electromagnetic field to be penetrated into the patient's head 11 for treating neurodegenerative disease present in the patient 11.

The EMF generation system 10 is structured to generate at least one time-varying electromagnetic field having a frequency of 64 MHz (from 10 to 400 MHz) and a specific absorption rate (SAR) of 0.6 W/kg (from 0.1 to 1.5 W/kg) to the brain tissue of a patient 11. Previous studies on primary human brain cultures at the Indiana University School of Medicine (IUSM) found that repeated electromagnetic field stimulation (REMFS) at a frequency of 64 MHz and a SAR at 0.6 W/kg decreased toxic amyloid-beta (AD) levels that cause AD. Furthermore, REMFS potentially decreases other protein accumulation such a Tau or alpha-synuclein protein due to activation of autophagy and protein degradation.

A frequency of 64 MHz (10 to 400 MHz) has good skin penetration and may easily pass through the various layers of the head, including skin, fat, dura, the cerebrospinal (CSF), and grey matter, and reach deeply into the brain tissues. The SAR of 0.6 W/kg may be achieved with lower power input and energy, decreasing the probability of thermal injury and enhancing the safety of these treatments.

The EMF generation system 10 includes a head-mounted antenna unit 12, a control system 14, and at least one coupling circuit 16 coupled between the head-mounted antenna unit 12 and the control system 14 as shown in FIG. 1A. The head-mounted antenna unit 12 is configured to be positioned on the patient's head 11 for individualized treatment of neurodegenerative disease. The control system 14 is communicable with the head-mounted antenna unit 12 via the coupling circuit 16 and provides feedback to an operator, such as a caregiver of the patient 11. The coupling circuit 16 causes the head-mounted antenna unit 12 to be responsive to signals provided by the control system 14.

The head-mounted antenna unit 12 includes a first ring 18, a second ring 20, a plurality of antenna legs 22 interconnecting the first ring 18 and the second ring 20, and at least one wire 23 coupled to at least one antenna leg 22 of the plurality of antenna legs 22 as shown in FIG. 2. The first ring 18 and the second ring 20, and the plurality of antenna legs 22 are each arranged about a central axis 13 of the head-mounted antenna unit 12. The head-mounted antenna unit 12 may also be referred to as a birdcage coil antenna 12.

In the illustrative embodiment shown in FIGS. 2 and 4, first ring 18 and the second ring 20 are each formed from a plurality of first and second ring segments 18S, 20S and capacitors 24 interconnecting such segments 18S, 20S. Accordingly, each antenna leg 22 is coupled to and interconnects corresponding first and second ring segments 18S, 20S. In other embodiments, the first ring 18 and the second ring 20 may each be continuous with the plurality of antenna legs 22 interconnecting the first and second rings 18, 20. In still other embodiments, the capacitors 24, or at least some of the capacitors 24, may be replaced with electrically insulators so as to electrically isolate the legs 22 from one another, or at least some of the legs 22 from others of the legs 22, so as to provide for driving of each of the legs 22, or at least some of the legs 22, with different levels of electrical power, e.g., different levels of signal amplitude.

The plurality of antenna legs 22 are distributed circumferentially about the central axis 13 as shown in FIGS. 2 and 4. As such, at least one antenna leg of the plurality of antenna legs 22 is spaced apart 180 degrees from another antenna leg of the plurality of antenna legs 22 relative to the central axis 13. In other embodiments, at least one antenna leg of the plurality of antenna legs is spaced apart between about 1 degree and about 359 degrees from another antenna leg of the plurality of antenna legs 22. When the head-mounted antenna unit 12 is positioned on the patient's head 11, the plurality of antenna legs 22 are distributed around the patients head 11 relative to the central axis 13. In the illustrative embodiment, the plurality of antenna legs 22 includes 16 antenna legs. In other embodiments, the plurality of antenna legs 22 may include less than 16 or more than 16 antenna legs.

The first ring 18, the second ring 20, and the plurality of antenna legs 22 cooperate to define a diameter D and a height H of the head-mounted antenna unit 12 as shown in FIGS. 2 and 4. In some embodiments, the diameter D may be about 12 inches and the height H may be about 12 inches. In other embodiments, the diameter D may be less than 12 inches, greater than 12 inches, and/or sized to permit a patient's head 11 to be positioned inside the head-mounted antenna unit 12. Additionally or alternatively, the height H may be less than 12 inches or greater than 12 inches. In some embodiments, the patient's head 11 may be no more than 6 inches away from a patient-facing surface 12S of the head-mounted antenna unit 12 such that a time-varying electromagnetic field may penetrate the patient's head 11 to a target depth from the head-mounted antenna unit 12. In other embodiments, the patient's head 11 may be between 0 inches and 6 inches away from the patient-facing surface 12S of the head-mounted antenna unit, or any specific distance or range of distances therein.

The at least one wire 23 is configured to electrically communicate at least one time-varying electrical signal from the control system 14 via the at least one coupling circuit 16 to at least one antenna leg 22 of the plurality of antenna legs 22 to produce at least one corresponding time-varying electromagnetic field to be directed into the patient's head 11. In some embodiments, the at least one wire 23 includes one wire 23 that operates each antenna leg of the plurality of antenna legs 22 simultaneously when the at least one time-varying electrical signal is received from the at least one coupling circuit 16. In other embodiments, the at least one wire 23 is configured to selectively operate one or more of the plurality of antenna legs 22 when the at least one time-varying electrical signal is received from the at least one coupling circuit 16.

In further embodiments, the at least one wire 23 could include a corresponding wire 23 for each of the plurality of antenna legs 22 to operate the plurality of antenna legs 22 separately, i.e., individually, and simultaneously. In other words, the at least one wire 23 could include a plurality of wires 23 configured to electronically communicate the at least one time-varying electrical signal received from the at least one coupling circuit 16 to their respective antenna leg 22 of the plurality of antenna legs 22. Additionally or alternatively, the at least one wire 23 could include a plurality of wires 23 that is less than the plurality of antenna legs 22 such that two or more antenna legs 22 are communicable with one wire 23 of the plurality of wires 23.

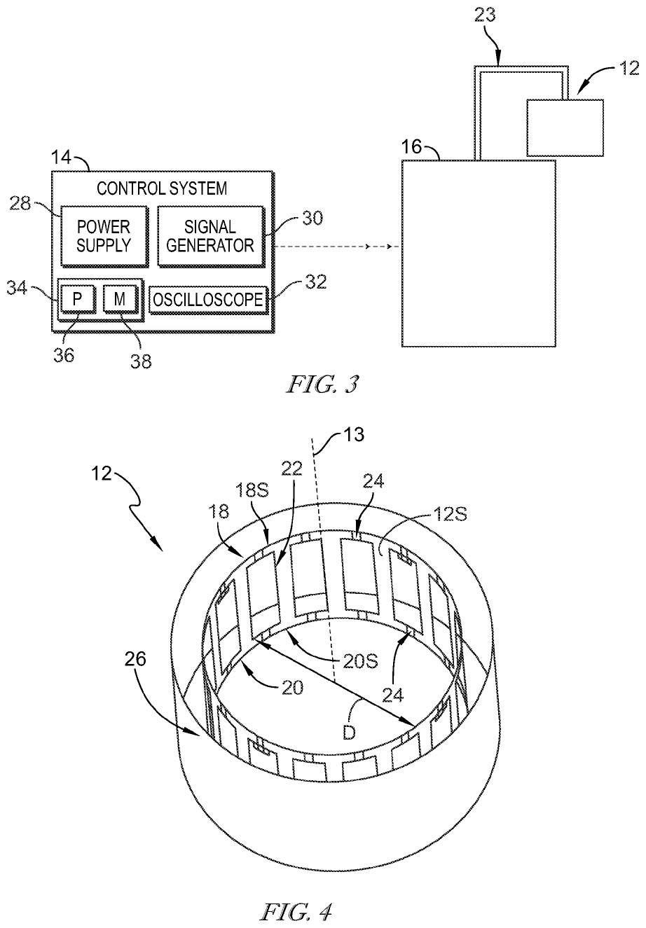

The control system 14 includes a power supply 28, a signal generator 30, an oscilloscope 32, and a controller 34 as shown in FIGS. 1A and 3. The power supply 28 provides power to the EMF generation system 10. The signal generator 30 is configured to generate one or more time-varying electrical signals. The oscilloscope 32 measures the frequency of the one or more time-varying electrical signals generated by the signal generator 30. The controller 34 is coupled to the signal generator 30.

The power supply 28 may be any suitable power supply or combination of power supplies for a medical instrument, including but not limited to, one or more batteries and/or one or more electrical outlet connections.

The signal generator 30 may be any signal generator capable of generating one or more time-varying electrical signals, including but not limited to a radiofrequency signal generator, a radiofrequency vector signal generator, an arbitrary function generator, an arbitrary waveform generator, a function generator, or a waveform generator. Likewise, the oscilloscope 32 may be any oscilloscope capable of measuring the frequency of the one or more time-varying electrical signals.

Alternatively, the functions of the signal generator 30 and/or the oscilloscope 32 may be implemented using one or more computer programs connected to input and output interfaces and communicable with other components of the EMF generation system, such as the head-mounted antenna unit 12, the at least one coupling circuit 16, the power supply 28, and/or the controller 34.

The controller 34 includes a processor 36 and a memory 38. The memory 38 stores instructions to, when executed by the processor 36, receive patient-specific data from user inputs and/or an internal or external network, determine a power of the one or more time-varying electrical signals based on the patient-specific data, and communicate the determined power of the one or more time-varying electrical signals to the signal generator 30 such that at least one time-varying electromagnetic field can penetrate the patient's head 11.

The instructions to determine a power, e.g., amplitude, of the one or more time-varying electrical signals may include further instructions to extract, determine, or identify measured parameters related to the physical characteristics of the patient's head 11 from the patient-specific data, calculate a target depth for the at least one time-varying electromagnetic field to penetrate the patient's head 11, and calculate a power of the one or more time-varying electrical signals, based on the measured parameter(s), that results in the at least one time-varying electromagnetic field to penetrate the patient's head 11 to the target depth. Calculating the target depth for the at least one time-varying electromagnetic field and/or the power of the at least one time-varying electrical signals may be based on a pre-determined specific absorption rate (SAR) for the at least one time-varying electromagnetic field, as discussed in further detail below. The measured parameters of the patient's head may include the thickness, volume, density, and location of one or more tissues or tissue layers of the patient's brain and of one or more bone, fluid and hair layers. The measured parameters of the patient's head further illustratively includes the conductivity, permittivity, and density of each of the foregoing layers. The conductivity, permittivity and density values of each of the layers are then provided as inputs to a conventional parametric model used to compute various dielectric properties of each of the layers for individual EMF frequencies. The dielectric properties of the various measured layers are then used, along with one or more additional variables to determine the power requirements of the electrical signals applied to the antenna legs 22, i.e., to determine the amplitudes of the electrical signals applied to the antenna legs 22 required to cause the resulting electromagnetic fields produced by the antenna legs 22 to penetrate the patients' head 11 to the target depth. Illustratively, the one or more additional variables may include, but are not limited to, the distance between the patient-facing surface 12S of the antenna unit 12 and the outer surface of the patient's head 11 and/or the distances between the patient-facing surfaces of each, or each group, of the antenna legs 22 and a respective portion of the outer surface of the patient's head 11, the size and/or structural configuration of the antenna legs 22, the frequency of the applied electrical signals, and the like.

The measured parameters may relate to the thickness, volume, density, location, conductivity, permittivity, and density of various features and/or layers of the patient's head and/or brain, including but not limited to, one or any combination of hair, skin, fat, connective tissue, skull bone, dura matter, arachnoid matter, pia matter, blood vessels, brain grey matter, brain white matter, and cerebrospinal fluid.

In some embodiments, one or more measured parameters may be extracted from the results of an MRI scan of the patient's head/brain. Alternatively or additionally, one or more of the measured parameters may be obtained via user inputs of previously measured parameters of the patient's head/brain. In further embodiments, one or more of the measured parameters may be communicated to the controller via a network connection, such as USB, WiFi, Bluetooth® or similar means.

The instructions to communicate the determined power to the signal generator 30 may include instructions to communicate a determined time period. The determined time period may be stored in the memory via user input or be pre-programmed into the memory. The determined time period may be at least one hour, although other time periods may alternatively be used.

The patient-specific data may be data of the patient's head and brain that was obtained from a magnetic resonance imaging (MRI) scan or other medical imaging method.

The control system 14 and/or electronic aspects of the EMF generation system 10 may be implemented by means of an analogue circuit, a digital circuit, or a computer arrangement with a processor instructed by a suitable computer program, or any combination thereof.

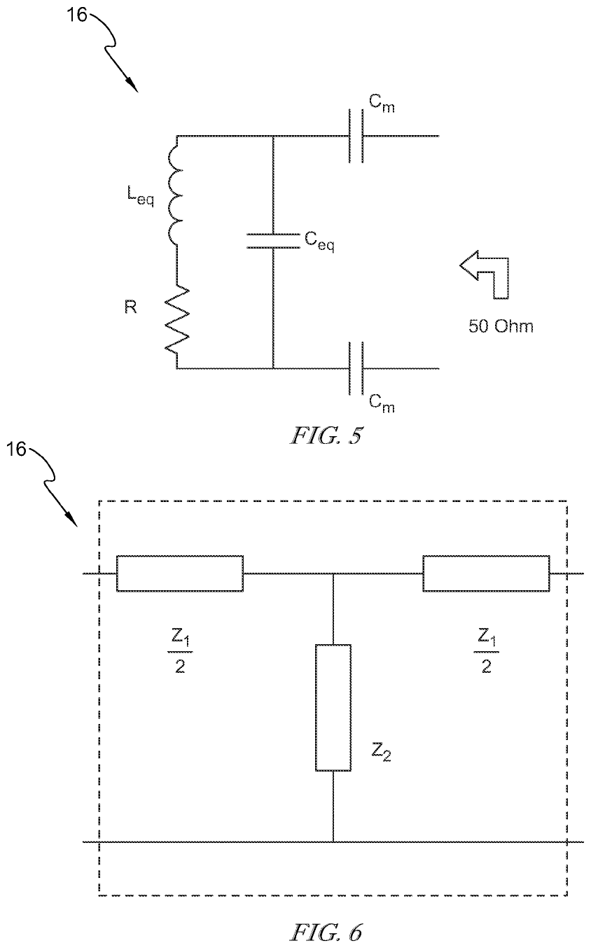

The at least one coupling circuit 16 is coupled between the signal generator 30 and at least one of the plurality of antenna legs 22. The at least one wire 23 is coupled between at least one of the plurality of antenna legs 22 and the at least one coupling circuit 16. Accordingly, each antenna leg 22 is responsive to the one or more time-varying electrical signals generated by the signal generator 30 via the at least one coupling circuit 16 to produce at least one corresponding time-varying electromagnetic field configured to be directed into the patient's head 11. A circuit diagram of the at least one coupling circuit 16 is shown in FIG. 5, and an equivalent circuit of impedance values Z1, Z2 is shown in FIG. 6.

In some embodiments, the at least one coupling circuit 16 may include one coupling circuit 16 communicable with each of the plurality of antenna legs 22. In other embodiments, the at least one coupling circuit 16 may include a plurality of coupling circuits 16 communicable with a corresponding antenna leg 22 of the plurality of antenna legs 22.

The at least one time-varying electrical signal and/or the at least one time-varying electromagnetic field may have a frequency of about 64 MHz. In other embodiments, the at least one time-varying electrical signal and/or the at least one time-varying electromagnetic field may have a frequency of about 10 MHz to about 400 MHz, or any specific frequency or range of frequencies therein. The frequency may be adjusted based on the target depth for the one or more time-varying electromagnetic fields. The power of the at least one time-varying electrical signal and/or the at least one time-varying electromagnetic filed may cause a specific absorption rate (SAR) of about 0.4 W/kg, to about 0.6 W/kg, or any specific SAR or range of SARs therein.

The target depth for the time-varying electromagnetic field may be the center of the patient's brain 11. In the embodiment illustrated in FIG. 2, for example, the target depth may be a point along the central axis 13 which bisects the height, H, of the antenna unit 12. In such instances, the patient's entire brain may receive the at least one time-varying electromagnetic field for treatment. In other embodiments, the target depth may be any point along the central axis 13. In still other embodiments, the target depth may be in one or more locations of the patient's brain or may vary around the circumference of the patient's head 11 in order to target specific area(s) of the brain which receive the at least one time-varying electromagnetic field. In such instances, antenna legs 22 which are positioned at or near the specific area(s) of the brain may be activated to generate the at least one time-varying electromagnetic field while other antenna legs 22 remain unactivated. The cylindrical design of the head-mounted antenna unit 12 allows for a homogenous SAR of the at least one time-varying electromagnetic field when all of the antenna legs 22 are activated.

In some embodiments, a single electrical signal may be supplied to all of the antenna legs 22 such that each antenna leg 22 generates an electromagnetic field with the same power/intensity. In other embodiments in which separate electrical signals are supplied to each, or to groups, of the antenna legs 22, the power, e.g., amplitude(s), of the electrical signals supplied to each, or to each group, of the antenna legs 22 may vary, depending on the distance between each, or each group, of the antenna legs 22 and the respective portion of the patient's head 11 or the target depth and/or upon one or more of the measured parameters of the corresponding portion(s) of the patient's head between each, or each group, of the antenna legs 22 and the target depth, to ensure that the electric fields produced by each, or each group, of the antenna legs 22 penetrates the patient's head to the target depth. One or more of the measured parameters described above, and thus the corresponding dielectric properties, may differ about the patient's head, and such differences may be compensated for by varying the power of the electrical signals supplied to each, or to each group, of the antenna legs 22 to ensure production of corresponding electromagnetic fields each with sufficient power to penetrate to the target depth of the patient's head 11.

During treatment, the patient 11 may be seated, as shown in FIG. 1, lying down, or standing. Treatment using the EMF generation system 10 may comprise a determined treatment time period of at least one hour a day for at least five days, however determined treatment time period may vary depending on the needs of the patient and assessment by a doctor or caregiver.

The EMF generation system 10 may further include a shell 26 arranged about the central vertical axis 13 and positioned radially outward of the head-mounted antenna unit 12 as shown in FIGS. 1 and 4. The shell 26 is made of a non-conductive material, such as rubber or plastic. Additionally or alternatively, the shell 26 may be arranged about the central axis 13 and positioned radially inward of the first ring 18, the second ring 20, and the plurality of antenna legs 22 to shield the patient 11 from direct contact with the head-mounted antenna unit 12.

Additionally or alternatively, the control system 14 may further include a temperature sensor (not shown) to monitor the patient's temperature. The temperature sensor may be communicable with the controller 34. Accordingly, the memory 38 may store instructions to, when executed by the processor 36, compare the temperature measured by the temperature sensor with a pre-determined temperature range and, if the measured temperature is outside of the pre-determined temperature, turn off the signal generator and/or the power supply to end treatment. Additionally, the memory 38 may store instructions to, when executed by the processor 36, display the measured temperature.

A method of treating neurodegenerative disease is also disclosed herein. The method includes inputting measured physical parameters into the memory 38, positioning the head-mounted antenna unit 12 at least partially about the patient's head 11, determining from the stored parameters of the patient's head 11 a power of electrical signals to be applied to at least one antenna leg 22 of the head-mounted antenna unit 12 to cause the at least one antenna leg 22 to produce at least one time-varying electromagnetic field to penetrate a target depth into the patient's head 1t, and applying the electrical signals with the power for the determined treatment time period.

The method may further include obtaining patient-specific data and determining the measured physical parameters from the patient-specific data. Obtaining patient-specific data may be from user inputs or network communications.

The EMF generation system 10 emulates a small-scale magnetic resonance imaging (MRI) machine, producing the same 64 MHz frequency at much lower operating power. In a computer simulation described below in Example 1, previously obtained EMF parameters of frequency and SAR found in previous studies were set to determine a power input for a simulated patient.

### Example

The following example provides further non-limiting disclosure of the EMF generation system 10.

In the present disclosure, a high-frequency simulation system (HFSS/EMPro) software is used to produce the birdcage structure of the head-mounted antenna unit 12 for the required EMF parameters.

The 64 MHz radiating frequency produces a scattering S₁₁ parameter of −15 dbs. A SAR of 0.6 W/kg may be observed when an input power of 100 W is applied. The dimensions of the RF birdcage coil 10 may include a 1-foot height and 12-inch diameter, which fits in wearable systems. The electric field and SAR radiate homogeneously over a simulated human head with good penetration into the brain, suggesting this to be an appropriate potential therapeutic strategy for AD.

Previous studies have demonstrated a method to prevent and treat aging and age-related diseases including repeated electromagnetic stimulation (REMFS). Such a method may be a potential therapy for AD. An exposure system which would provide a SAR of 0.4 to 0.6 W/kg uniformly to a human brain has been developed. A mathematical and simulation EMF model was developed in a multilayer human head to find the exposure parameters for a device which provides the SAR was found to decrease Aβ levels in human primary brain cell cultures and AD mouse model studies. The parameters determined from the mathematical and simulation EMF model were validated and reduced into a practical model to develop the EMF generation system 10 using a specific anthropomorphic mannequin (SAM). The EMF generation system 10 includes a power generator and a helmet antenna. The EMF generation system 10 covers a range of radio frequencies between 1 MHz and 300 MHz. The EMF generation system 10 is also referred to as a REMFS exposure system, and the system and method of use thereof may be utilized for preventing and treating AD. The EMF generation system 10 may also decrease Aβ in human neuron. REMFS activates biological pathways that degrade the toxic Aβ that causes AD.

The simulated EMF generation system 10 is designed to provide a SAR of 0.6 W/Kg and a frequency of 64 MHz for a proper time constant that produces a small change in temperature within a fraction of m^{o}K. With the circuit diagram shown in FIG. 5, the radiating frequency, f₀ is given by:

$${f}_{0} = \frac{1}{2 π \sqrt{{L}_{eq} {C}_{eq}}} \qquad ( 1 )$$

When L_{eq} is chosen to be 174.7 nH, and C_{eq} to be 35.6 pF, the EMF birdcage wearable device 10 radiates at f₀=64 MHz.

The controlling equations for these capacitances and inductances are given as:

$${C}_{eq} = \frac{2 α {β}^{2} {C}_{t}}{N} \qquad ( 2 )$$

choosing Cₜ=142.4 pF, Ceq=35.6 pF.

The values of L_{rings} and Lₛₜᵣᵢₚ may be estimated from the equations:

$${L}_{end rings} = \frac{{μ}_{0} l}{2 π} ( ln ( \frac{2 l}{a} ) - 1 ) \qquad ( 3 )$$

$${L}_{strip} = \frac{{μ}_{0} l}{2 π} ( ln ( \frac{2 l}{w} ) + \frac{1}{2} ) \qquad ( 4 )$$

For the model of the circuit diagram shown in FIG. 5,

$${L}_{eq} = \frac{{Z}_{in}}{j ω} \qquad ( 5 )$$

$${Z}_{in} = \frac{{Z}_{c} sin h ( \frac{N λ}{2} )}{2 sin h ( \frac{λ}{2} ) sin h [ ( N - 1 ) ( \frac{λ}{2} ) ]} \qquad ( 6 )$$

$${Z}_{c} = [ {( \frac{{Z}_{1}}{2} )}^{2} + {Z}_{1} {Z}_{2} ] \qquad ( 7 )$$

$$λ = cos {h}^{- 1} ( 1 + \frac{{Z}_{1}}{2 {Z}_{2}} ) \qquad ( 8 )$$

Given a number of rings N=16, the spatial phase factor φ, is determined from

$$ϕ = \frac{π}{N} \qquad ( 9 )$$

The spatial phase factor φ, in the present example equals 196.35 m rad.

The value of α that may be used in equation (3) above can be determined from:

$$α = \{ \begin{matrix}1 , & for low pass \\ 2 {sin}^{2} ϕ , & for high pass\end{matrix} \qquad ( 10 )$$

with high pass filter consideration, α=2 sin² φ, and where φ is given in equation (9) above.

The value of β as given in equation (2) above can be determined from

$$β = \frac{1}{2} {\sum }_{n = 1}^{N} \vert cos [ \frac{π}{n} ( 2 n + 1 ) ] \vert \qquad ( 11 )$$

with N=16. In such scenarios, the value of β is 5.126. Table 1 presents the relationship between N and β.

TABLE 1
β Values as a Function of the Birdcage Legs Number N

| N | 4 | 8 | 12 | 16 |
|---|---|---|---|---|
| β | 1.414 | 2.613 | 3.863 | 5.126 |

The above equations will lead to values: Cₜ=142.4 pF, C_{eq}=35.6 pF, L_{end rings}=30 nH, and Lₛₜᵣᵢₚ=208 nH.

Considering the resistance of the cage coil, R_{coil} is given by:

$${R}_{coil} = 2 π BL \qquad ( 12 )$$

where B is the 3 dB frequency and considering 3 dbs=25 KHz.

The quality factor of the coil may be estimated from the following equations:

$${Q}_{empty} = \frac{{f}_{c}}{{f}_{2} - {f}_{1}} = \frac{2 π {f}_{0} L}{{R}_{coil}} \qquad ( 13 )$$

$$Q = \frac{{Q}_{empty}}{{Q}_{load}} = \frac{{R}_{coil} + {R}_{samp}}{{R}_{coil}} \qquad ( 14 )$$

$$\frac{1}{2} {C}_{m} = \sqrt{\frac{{C}_{eq}}{2 π {f}_{0} {Q}_{load} 50}} \qquad ( 15 )$$

Following the above equations, Cₘ may be estimated to be 20.37 pF.

The change in temperature when the system reaches the 0.6 W/Kg SAR value is estimated from:

$$Δ {T}_{R} = \frac{{c}_{p} Δ T}{Δ SAR} \qquad ( 16 )$$

where ΔT_{R} is the rise time, ΔT is the change in temperature based on the value of ΔSAR (which is 0.6 SAR in this case), and cₚ is the specific heat capacity.

The rise time, ΔT_{R} is given by:

$$Δ {T}_{R} = \sqrt{2 π} τ \qquad ( 17 )$$

where τ is the time constant given by:

$$τ = \frac{L}{R} = RC \qquad ( 18 )$$

The specific heat capacity in the present example is taken as cₚ=3.46 kJ.

The above equations estimate the time constant and temperature values equal: ΔT=0.25 m⁰K and ΔT_{R}=1.44 s.

High-frequency simulation system (HFSS/EMPro) software was used in simulating the EMF generation system 10 to estimate the SAR distribution inside the head-mounted antenna unit 12. A diameter of 12-inches and a height of 1-foot was estimated to achieve the required SAR of 0.6 W/kg inside the birdcage coil 10. An electric field (E-field) 12 was found to be relatively homogeneous inside the birdcage coil 10. The head-mounted antenna unit 12 was designed with a distance of 16 and 5 mm between wires. A magnetic field (H-field) 14 was proven to have a wider shield with the E-field 12 distribution. Displaced conductors more than 10 mm cause the H-field 14 and accordingly electromagnetic energy to escape from the head-mounted antenna unit 12 structure.

The distance between wires was critical in controlling the E-field 12 distribution. A larger distance may cause the escape of the E-field 12 outside the birdcage coil 10. Matlab was used for the mathematical model described above with the ratio between the head-mounted antenna unit 12 radius to shield of 4:5, the shield radius was equal to 1.25× the birdcage radius, and the shield radius was near 6-inch diameter.

The value of H=1.2M occurred almost uniformly inside the coil; this result matches with the Matlab simulation, with the H-field 14, needed to be 1.19 M Amps/Meter, with energy consumption (input power) reaching near 100 W in a short time.

The simulation of the present example showed that the magnetic energy impacting the SAR value is focused inside of head-mounted antenna unit 12 with H values near 1.25×10⁶ A/m. This makes B values near 1.5 T, close to the B value generated from the MRI coils. The data shows that the magnetic field quickly attenuates outside the birdcage coil 10. The value of H above the birdcage coil 10 was near 22 KA/m, making the B value near 27 mT. The high localized fields near the end rings are of narrow regions and not propagating to the inside. These are result from boundary values of the fields near the rings.

A EMF generation system 10 may produce the required EMF parameters of a SAR of 0.6 W/kg (0.1 to 1.5 W/kg), and a frequency of 64 MHz (10 to 400 MHz) following the preliminary biological data presented in previous studies. The HFSS simulation results demonstrate that the head-mounted antenna unit 12 dimensions are suitable as a wearable device for AD treatments. The simulation demonstrates homogeneous field distribution for the E-field 12 and the H-field 14 in the middle of the head-mounted antenna unit 12, suggesting uniform power density applied to the simulated head-mounted antenna unit 12.

With no curative pharmacological agents available in the market, recent efforts from the joint medical/engineering team at Indiana University Purdue University Indianapolis (IUPUI) have investigated the use of a non-ionizing, non-invasive, low power, low frequency electromagnetic field (EMF) for the treatment of AD patients via radiofrequency (RF) devices in efforts to decreasing the toxic amyloid-beta (AD) levels, which is considered to be the cause of AD. Also, the fact that it is a low energy, non-ionizing EMF frequency makes it safe for human treatments. Furthermore, it has been used safely by magnetic resonance imaging (MRI) devices for decades. In the present disclosure, a high-frequency simulation system (HFSS) was utilized to produce the antenna parameters and the field distribution following the preliminary results reported in previous studies.

The EMF generation system 10 of the present disclosure is based on the medical diagnostic MRI system for high-quality anatomical images, which includes highly uniform magnetic and electric fields. The EMF generation system 10 also features a high signal-to-noise ratio, design flexibility, and the ability to be designed for multi-resonance operation. The EMF generation system 10 may be modeled as a closed ladder network composed of identical cascaded segments of inductive and capacitive elements.

The present disclosure applied multiple closed current loops in the EMF generation system 10 that may give equivalencies to low pass, band pass, and high pass filter structures. The multiple cascaded identical segments result in multiple resonance frequencies, also known as resonance modes. This depends on the number of legs 22, the inductance of each leg 22 and end ring segments 18, 20, and the lumped capacitance of both the leg 22 and the end ring 18, 20. Most existing solutions involve complex mathematical formulation or are limited to the determination of the resonance characteristics of the EMF generation system 10. In practice, this may require impedance matching circuits with the EMF generation system 10 following the mathematical model done in a recent study.

The following numbered clauses are contemplated and non-limiting:

1. An electromagnetic field (EMF) generation system for treatment of neurodegenerative disease in a patient, the EMF generation system comprising: a head-mounted antenna unit having at least one ring arranged about a central axis of the head-mounted antenna unit and a plurality of antenna legs coupled to the at least one ring.

2. The EMF generation system of clause 1, any suitable clause, or any suitable combination of clauses, wherein a first antenna leg of the plurality of antenna legs is spaced apart 180 degrees from a second antenna leg of the plurality of antenna legs relative to the central axis such that the first antenna leg and the second antenna leg are arranged around a patient's head when the head-mounted antenna unit is positioned on the patient's head.

3. The EMF generation system of clauses 1 and 2, any suitable clause, or any suitable combination of clauses, wherein the EMF generation system further comprises a signal generator configured to generate one or more time-varying electrical signals.

4. The EMF generation system of clauses 1-3, any suitable clause, or any suitable combination of clauses, wherein the EMF generation system further comprises at least one coupling circuit coupled between the signal generator and at least one of the plurality of antenna legs such that each antenna leg is responsive to the one or more time-varying electrical signals generated by the signal generator to produce at least one corresponding time-varying electromagnetic field configured to be directed into the patient's head.

5. The EMF generation system of clauses 1-4, any suitable clause, or any suitable combination of clauses, wherein the EMF generation system further comprises at least one controller coupled to the signal generator and having at least one processor and at least one memory storing instructions to, when executed by the at least one processor, determine a power of the time-varying electrical signals based on measured parameters related to physical characteristics of the patient's head which will result in the at least one time-varying electromagnetic field penetrating the patient's head to a target depth.

6. The EMF generation system of clauses 1-5, any suitable clause, or any suitable combination of clauses, wherein the at least one memory further includes instructions to, when executed by the at least one processor, receive user inputs to obtain the measured parameters related to the physical characteristics of the patient's head.

7. The EMF generation system of clauses 1-6, any suitable clause, or any suitable combination of clauses, wherein the plurality of antenna legs includes sixteen antenna legs.

8. The EMF generation system of clauses 1-7, any suitable clause, or any suitable combination of clauses, wherein the at least one time-varying electromagnetic field has a frequency of about 10 to about 400 MHz.

9. The EMF generation system of clauses 1-8, any suitable clause, or any suitable combination of clauses wherein the frequency is about 64 MHz.

10. The EMF generation system of clauses 1-9, any suitable clause, or any suitable combination of clauses wherein the power of the time-varying electrical signals causes a specific absorption rate of about 0.4 to about 0.6 W/kg.

11. The EMF generation system of clauses 1-10, any suitable clause, or any suitable combination of clauses wherein the measured parameters includes at least one of the conductivity, permittivity, and density of one or more features of the patient's head, wherein dielectric properties of each of the features are determined using the at least one of the conductivity, permittivity and density values of each of the features for individual EMF frequencies, and wherein the memory further includes instructions to, when executed by the processor, determine the power of the time-varying electrical signals with at least the dielectric properties.

12. A head-mounted antenna unit for treatment of neurodegenerative disease in a patient, the head-mounted antenna unit comprising: at least one ring arranged about a central axis.

13. The head-mounted antenna unit of clause 12, any suitable clause, or any suitable combination of clauses, wherein the head-mounted antenna unit further comprises a plurality of antenna legs coupled to and extending axially away from the at least one ring.

14. The head-mounted antenna unit of clauses 12 and 13, any suitable clause, or any suitable combination of clauses, wherein a first antenna leg of the plurality of antenna legs is spaced apart 180 degrees from a second antenna leg of the plurality of antenna legs relative to the central axis such that the first antenna leg and the second antenna leg are arranged around a patient's head when the head-mounted antenna unit is positioned on the patient's head.

15. The head-mounted antenna unit of clauses 12-14, any suitable clause, or any suitable combination of clauses, wherein the head-mounted antenna unit further comprises at least one wire coupled to at least one antenna leg of the plurality of antenna legs, wherein the at least one wire is configured to electrically communicate with a signal generator to produce at least one corresponding time-varying electromagnetic field configured to be directed into the patient's head from at least one antenna leg of the plurality of antenna legs.

16. The head-mounted antenna unit of clauses 12-15, any suitable clause, or any suitable combination of clauses, wherein the plurality of antenna legs includes sixteen antenna legs.

17. The head-mounted antenna unit of clauses 12-16, any suitable clause, or any suitable combination of clauses, wherein the at least one time-varying electromagnetic field has a frequency of about 10 to about 400 MHz.

18. The head-mounted antenna unit of clauses 12-16, any suitable clause, or any suitable combination of clauses, wherein the frequency is about 64 MHz.

19. The head-mounted antenna unit of clauses 12-18, any suitable clause, or any suitable combination of clauses, wherein the at least one wire includes a first wire and a second wire, and wherein the first wire is coupled with the first antenna leg and the second wire is coupled with the second antenna leg.

20. The head-mounted antenna unit of clauses 12-19, any suitable clause, or any suitable combination of clauses, wherein the first wire and the second wire are configured to selectively operate the first antenna leg and the second antenna leg.

21. A method of treating neurodegenerative disease for a particular patient, the method comprising inputting measured physical parameters of a patient's head into a memory of a treatment system.

22. The method of clause 21, any suitable clause, or any suitable combination of clauses, wherein the method further comprises positioning an antenna unit at least partially about the patient's head including a plurality of antenna legs.

23. The method of clauses 21 and 22, any suitable clause, or any suitable combination of clauses, wherein the method further comprises determining from the stored parameters of the patient's head a power of electrical signals to be applied to the at least one antenna leg of the antenna unit to cause the at least one antenna leg to produce at least one time-varying electromagnetic field to penetrate a target depth into the patient's skull.

24. The method of clauses 21-23, any suitable clause, or any suitable combination of clauses, wherein the method further comprises applying the electrical signals with the power for a determined treatment time period.

25. The method of clauses 21-24, any suitable clause, or any suitable combination of clauses, wherein the method further comprises obtaining measurements of the patient's head and determining the measured physical parameters from the measurements of the patient's head.

26. The method of clauses 21-25, any suitable clause, or any suitable combination of clauses, wherein the measured parameters includes at least one of the conductivity, permittivity, and density of one or more features of the patient's head, wherein dielectric properties of each of the layers are determined using the at least one of the conductivity, permittivity and density values of each of the layers for individual EMF frequencies, and wherein the method further comprises determining the power of the time-varying electrical signals with at least the dielectric properties.

27. The method of clauses 21-26, any suitable clause, or any suitable combination of clauses, wherein the target depth into the patient's skull is the center of the patient's skull.

28. The method of clauses 21-27, any suitable clause, or any suitable combination of clauses, wherein the antenna unit is a birdcage coil antenna.

29. The method of clauses 21-28, any suitable clause, or any suitable combination of clauses, wherein the at least one time-varying electromagnetic field has a frequency of about 64 MHz.

30. The method of clauses 21-29, any suitable clause, or any suitable combination of clauses, wherein the power of the time-varying electrical signals causes a specific absorption rate of about 0.4 to about 0.6 W/kg.

While the concepts of the present disclosure are susceptible to various modifications and alternative forms, specific exemplary embodiments of the disclosure have been shown by way of example in the drawings. It should be understood, however, that there is no intent to limit the concepts of the present disclosure to the particular disclosed forms; the intention is to cover all modifications, equivalents, and alternatives falling within the spirit and scope of the invention as defined by the claims. Although this disclosure refers to specific embodiments, it will be understood by those skilled in the art that various changes in form and detail may be made without departing from the subject matter set forth in the accompanying claims.

1. An electromagnetic field (EMF) generation system for treatment of neurodegenerative disease in a patient, the EMF generation system comprising:
a head-mounted antenna unit having at least one ring arranged about a central axis of the head-mounted antenna unit and a plurality of antenna legs coupled to the at least one ring, wherein a first antenna leg of the plurality of antenna legs is spaced apart 180 degrees from a second antenna leg of the plurality of antenna legs relative to the central axis such that the first antenna leg and the second antenna leg are arranged around a patient's head when the head-mounted antenna unit is positioned on the patient's head,
a signal generator configured to generate one or more time-varying electrical signals,
at least one coupling circuit coupled between the signal generator and at least one of the plurality of antenna legs such that each antenna leg is responsive to the one or more time-varying electrical signals generated by the signal generator to produce at least one corresponding time-varying electromagnetic field configured to be directed into the patient's head, and
at least one controller coupled to the signal generator and having at least one processor and at least one memory storing instructions to, when executed by the at least one processor, determine a power of the time-varying electrical signals based on measured parameters related to physical characteristics of the patient's head which will result in the at least one time-varying electromagnetic field penetrating the patient's head to a target depth.

2. The EMF generation system of claim 1, wherein the at least one memory further includes instructions to, when executed by the at least one processor, receive user inputs to obtain the measured parameters related to the physical characteristics of the patient's head.

3. The EMF generation system of claim 1, wherein the plurality of antenna legs includes sixteen antenna legs.

4. The EMF generation system of claim 1, wherein the at least one time-varying electromagnetic field has a frequency of about 10 to about 400 MHz.

5. The EMF generation system of claim 4, wherein the frequency is about 64 MHz.

6. The EMF generation system of claim 1, wherein the power of the time-varying electrical signals causes a specific absorption rate of about 0.4 to about 0.6 W/kg.

7. The EMF generation system of claim 1, wherein the measured parameters includes at least one of the conductivity, permittivity, and density of one or more features of the patient's head, wherein dielectric properties of each of the one or more features are determined using the at least one of the conductivity, permittivity, and density values of each of the one or more features for individual EMF frequencies, and wherein the memory further includes instructions to, when executed by the at least one processor, determine the power of the time-varying electrical signals with at least the dielectric properties.

8. A head-mounted antenna unit for treatment of neurodegenerative disease in a patient, the head-mounted antenna unit comprising:
at least one ring arranged about a central axis,
a plurality of antenna legs coupled to and extending axially away from the at least one ring, wherein a first antenna leg of the plurality of antenna legs is spaced apart 180 degrees from a second antenna leg of the plurality of antenna legs relative to the central axis such that the first antenna leg and the second antenna leg are arranged around a patient's head when the head-mounted antenna unit is positioned on the patient's head, and
at least one wire coupled to at least one antenna leg of the plurality of antenna legs, wherein the at least one wire is configured to electrically communicate with a signal generator to produce at least one corresponding time-varying electromagnetic field configured to be directed into the patient's head from at least one antenna leg of the plurality of antenna legs.

9. The head-mounted antenna unit of claim 8, wherein the plurality of antenna legs includes sixteen antenna legs.

10. The head-mounted antenna unit of claim 8, wherein the at least one time-varying electromagnetic field has a frequency of about 10 to about 400 MHz.

11. The head-mounted antenna unit of claim 10, wherein the frequency is about 64 MHz.

12. The head-mounted antenna unit of claim 8, wherein the at least one wire includes a first wire and a second wire, and wherein the first wire is coupled with the first antenna leg and the second wire is coupled with the second antenna leg.

13. The head-mounted antenna unit of claim 12, wherein the first wire and the second wire are configured to selectively operate the first antenna leg and the second antenna leg.

14. A method of treating neurodegenerative disease for a particular patient, the method comprising
inputting measured physical parameters of a patient's head into a memory of a treatment system,
positioning an antenna unit at least partially about the patient's head including a plurality of antenna legs,
determining from the measured physical parameters of the patient's head a power of electrical signals to be applied to at least one antenna leg of the antenna unit to cause the at least one antenna leg to produce at least one time-varying electromagnetic field to penetrate a target depth into the patient's head, and
applying the electrical signals with the power for a determined treatment time period.

15. The method of claim 14, wherein the method further comprises obtaining measurements of the patient's head and determining the measured physical parameters from the measurements of the patient's head.

16. The method of claim 14, wherein the measured physical parameters includes at least one of the conductivity, permittivity, and density of one or more features of the patient's head, wherein dielectric properties of each of the one or more features are determined using the at least one of the conductivity, permittivity, and density values of each of the one or more features for individual EMF frequencies, and wherein the method further comprises determining the power of the time-varying electrical signals with at least the dielectric properties.

17. The method of claim 14, wherein the target depth into the patient's head is the center of the patient's head.

18. The method of claim 14, wherein the antenna unit is a birdcage coil antenna.

19. The method of claim 14, wherein the at least one time-varying electromagnetic field has a frequency of about 64 MHz.

20. The method of claim 14, wherein the power of the electrical signals causes a specific absorption rate of about 0.4 to about 0.6 W/kg.
